(19) United States
(12) Patent Application Publication
(10) Pub. No.: US 2023/0016556 A1
(43) Pub. Date: Jan. 19, 2023

(54) Photobiomodulation System for Improving Athletic Performance

(71) Applicant: Lew LIM, Toronto (CA)

(72) Inventor: Lew LIM, Toronto (CA)

(21) Appl. No.: 17/757,299

(22) PCT Filed: Jan. 26, 2021
(86) PCT No.: PCT/IB2021/050569

§ 371 Date: Jun. 13, 2022

Related U.S. Application Data
(60) Provisional application No. 62/969,779, filed on Feb. 4, 2020.

(51) Int. Cl. A61N 5/06

(52) U.S. Cl. CPC A61N 5/0622; A61N 2005/0659; A61N 2005/0663; A61N 2005/0626; A61N 2005/0647

(57) ABSTRACT

A self-administrable system for improving athletic performance of a subject, the said system comprising:
configured irradiation units comprising a first, a second, a third and a fourth configured irradiation unit, wherein:
(A) said first configured irradiation unit is positioned to direct light energy to a first region of the brain comprising the OZ position of the primary visual cortex;
(B) said second configured irradiation unit is positioned to direct light energy to a second region of the brain comprising the CZ position of the primary sensorimotor cortex;
(C) said third configured irradiation unit is positioned to direct light energy to a third region of the brain comprising the C3 position of the primary sensorimotor cortex; and
(D) said fourth configured irradiation unit is positioned to direct light energy to a fourth region of the brain comprising the C4 position of the primary sensorimotor cortex.

### FIELD OF THE INVENTION

The present invention relates to photobiomodulation, and more specifically, to a system and method of photobiomodulation for improving athletic performance.

### BACKGROUND ART

Spatial Perception and Positioning Sense

Spatial perception is a person's ability to be aware of his or her relationships with the surrounding environment and with himself or herself.

Spatial perception along with positioning sense are highly relevant to sports and athletic performance. For example, professional soccer players generally have a higher than normal perception for body kinematics which helps them perform in response to complex, dynamic visual scenes. In figure skating, this ability is trained at the highest level. As another example, a fencers' achievement level is highly correlated with spatial perception-related skills such as visual discrimination, visual-spatial relationships, visual sequential memory, narrow attentional focus and visual information processing.

This even applies to eSports, sports competitions using video games. Studies have found that individuals who play eSports shooting games at a high level have better developed spatial perceptual skills such as faster and more accurate performance in a peripheral and identification task.

Visual Processing

Visual processing refers to the brain's ability to use and interpret visual information from the world around us.

Visual processing is often coupled with spatial perception. A study found that cricket players who were faster at picking up information from briefly presented visual displays were significantly better batsmen in an actual game. Another study has shown that visual processing skills would be important for hockey players' actual performance on the ice.

Motor Skills

A motor skill is a learned ability to cause a predetermined movement outcome with maximum certainty. Motor learning is the relatively permanent change in the ability to perform a skill as a result of practice or experience. Motor performance is an act of executing a motor skill. The goal of motor skills is to optimize the ability to perform a skill successfully and precisely, with reduce the energy consumption. This can be achieved with continuous practice of a specific motor skill.

There are many different types of motor skills used in sports, including running, jumping, hopping, galloping, rolling, leaping, dodging, sliding, throwing, catching, kicking, striking, dribbling, balancing, twisting, turning and bending. A good example is gymnastics where many of the above motor skills must be performed at a high level.

Hand-Eye Coordination

Hand-eye coordination is the coordinated control of eye movement with hand movement and the processing of visual input to guide reaching and grasping along with the use of proprioception of the hands to guide the eyes.

Hand-eye coordination is well known to be tied to athletic performance. In sports, tracking performance is improved if the eyes and hands follow the same spatial trajectory, but better still if the eye leads the hand by about 75 to 100 ms. This suggests that information from the ocular control system quickly feeds into the manual motor control system to assist its tracking and muscular execution, supported by spatial perception and other functions.

Reflex Conditioning

Reflexes refer to actions performed in response to a stimulus and without deliberate thought. A conditioned reflex, also known as a conditioned response, is an acquired response in which the subject learns to associate a previously unrelated neutral stimulus with a different stimulus that elicits a specific reaction.

Well-conditioned reflexes are important to many athletes who need to react to a stimulus quickly and without being slowed down by deliberations. For example, sprinters must be conditioned to react to the sound of a starting pistol (the stimulus) in less than 200 ms by starting their sprint or they will be at an immediate disadvantage to their competitors. As another example, a tennis player returns incoming shots without deliberate thoughts; and a well-conditioned player would quickly judge the flight of the ball and return it with the right timing and strength.

Movement-Related Plasticity

Plasticity refers to the brain's ability to change and adapt to environmental influences and training/learning. Professional athletes or those who wish to be experts need to train consistently and with specificity, to drive brain plasticity and strengthen muscle memory.

Photobiomodulation

Photobiomodulation (PBM), also known as low-level light therapy (LLLT), is a biostimulation technique that has shown promise in treating some medical conditions, including dementia and Alzheimer's Disease. PBM can be used to stimulate the brain to extract the desirable outcomes.

The biochemical mechanism of PBM interaction can be classified into direct and indirect effects. The direct effects include increasing the activity of ion channels such as the Na+/K+ ATPase and the indirect effects include regulating important secondary messengers such as calcium, cyclic adenosine monophosphate (cAMP) and reactive oxygen species (ROS)—all of which result in diverse biological cascades. These biological cascades lead to effects such as the maintenance of homeostasis and activating protective, anti-oxidant and proliferative gene factors, as well as the systematic responses, such as cerebral blood flow, which is deficient in neurocognitive disorders.

The most well investigated mechanism of action of PBM is its fundamental effect on mitochondrial function. PBM has been demonstrated to increase the activity of complexes in the electron transport chain of mitochondria, comprising complex I, II, III, IV and succinate dehydrogenase. In complex IV, the enzyme cytochrome c oxidase (CCO), functions as photo acceptor as well as transducer. CCO specifically accepts and transduces light in the red (620-700 nm) and the near-infrared (780-1110 nm) wavelengths of lights which can be processed in PBM. The process increases the amount of ATP produced, as well as cyclic adenosine monophosphate (cAMP) and reactive oxygen species (ROS). The increase in ATP increases the activity of ion channels regulating cAMP and calcium, which results in the stimulation of diverse biological cascades and activate up to 110 genes for transcription, which lead to healing and recovery activities and the prolongation of the production of energy by the mitochondria. One of the most prominent responses to PBM is the activation of sodium pumps and the Na+/K+ ATPase, which leads to greater membrane stability and resistance to depolarization.

In addition to increasing the levels of ATP and cAMP, it has been observed that PBM results in an increase in nitric oxide (NO) levels. NO is dissociated from CCO when photons are absorbed by CCO. The dissociation of NO from CCO leads to the enhancement of ATP production and acts as a vasodilator as well as a dilator of lymphatic flow, activating a number of beneficial cellular pathways.

It would be advantageous to use PBM to improve athletic performance, which is achievable through improved spatial perception, visual processing, hand-eye coordination, reflex conditioning, motor skills and movement-related plasticity.

### SUMMARY OF THE INVENTION

In one aspect, the present invention provides a self-administrable system for improving athletic performance of a subject, said system comprising: configured irradiation units comprising or consisting of a first, a second, a third and a fourth configured irradiation unit, each of said first, second, third and fourth configured irradiation units comprising a portable hollow casing having fixed dimensions, a sized internal spatial volume and an external surface configuration suitable for application to the skull, said portable hollow casing of each configured irradiation unit being comprised of:

(i) a light energy transmitting material which forms at least a portion of the configured external surface for said hollow casing of each configured irradiation unit; and

(ii) at least one light generating unit housed and contained within said internal spatial volume of said hollow casing of each configured irradiation unit and which is capable of generating light energy of at least one preselected wavelength selected from the group consisting of near infrared light wavelengths and visible red light wavelengths, at a predetermined energy intensity, for a preset time duration, and at a predetermined pulse frequency, collectively on-demand sufficient to penetrate through the skull and to pass into the brain,

whereby said first, second, third and fourth configured irradiation units can emit light energy after application to the skull and achieve passage of said emitted light energy through the skull into at least one portion of the brain in-vivo;

a frame adapted for support of said first, second, third and fourth configured irradiation units and for at will placement of said light transmitting external surface of said first, second, third and fourth configured irradiation units at a fixed position and desired irradiation direction on the skull;

a portable controller assembly able to control on-demand delivery of light energy from said configured irradiation units into at least one portion of the brain in-vivo, said controller assembly including:

(a) a portable and replenishable power source of on-demand direct electrical current,

(b) a central processing unit for controlling and directing the flow of such direct electrical current,

(c) at least one connector in electrical communication with the power source for on-demand conveyance of direct electrical current to the central processing unit, and

(d) at least one connector in electrical communication with the configured irradiation units for on-demand conveyance of direct electrical current from said central processing unit to said light generating units; wherein:

(A) said first configured irradiation unit is positioned to direct light energy to a first region of the brain comprising the OZ position of the primary visual cortex;

(B) said second configured irradiation unit is positioned to direct light energy to a second region of the brain comprising the CZ position of the primary sensorimotor cortex;

(C) said third configured irradiation unit is positioned to direct light energy to a third region of the brain comprising the C3 position of the primary sensorimotor cortex; and

(D) said fourth configured irradiation unit is positioned to direct light energy to a fourth region of the brain comprising the C4 position of the primary sensorimotor cortex.

In another aspect, the present invention provides a self-administrable method for improving athletic performance of a subject, said method comprising the steps of:

obtaining a light energy-emitting apparatus comprised of:

configured irradiation units comprising or consisting of a first, a second, a third and a fourth configured irradiation unit, each of said first, second, third and fourth configured irradiation units comprising a portable hollow casing having fixed dimensions, a sized internal spatial volume and an external surface configuration suitable for application to the skull, said portable hollow casing of each configured irradiation unit being comprised of:

(i) a light energy transmitting material which forms at least a portion of the configured external surface for said hollow casing of each configured irradiation unit; and

(ii) at least one light generating unit housed and contained within said internal spatial volume of said hollow casing of each configured irradiation unit and which is capable of generating light energy of at least one preselected wavelength selected from the group consisting of near infrared light wavelengths and visible red light wavelengths, at a predetermined energy intensity, for a preset time duration, and at a predetermined pulse frequency, collectively on-demand sufficient to penetrate through the skull and to pass into the brain,

whereby said first, second, third and fourth configured irradiation units can emit light energy after application to the skull and achieve passage of said emitted light energy through the skull into at least one portion of the brain in-vivo;

a frame adapted for support of said first and second configured irradiation units and for at will placement of said light transmitting external surface of said first and second configured irradiation units at a fixed position and desired irradiation direction on the skull;

a portable controller assembly able to control on-demand delivery of light energy from said configured irradiation lenses into at least one portion of the brain in-vivo, said controller assembly including:

(a) a portable and replenishable power source of on-demand direct electrical current,

(b) a central processing unit for controlling and directing the flow of such direct electrical current,

(c) at least one connector in electrical communication with the power source for on-demand conveyance of direct electrical current to the central processing unit, and

(d) at least one connector in electrical communication with the configured irradiation units for on-demand conveyance of direct electrical current from said central processing unit to said light generating units;

placing a transparent external surface of said first, second, third and fourth configured irradiation units at a desired fixed position adjacent to the skull of a subject such that light energy emitted by said first, second, third and fourth configured irradiation units will penetrate through the subject's skull and pass into at least one portion of the brain in-vivo; and

causing said light generating units of said positioned configured irradiation units to generate light energy of at least one preselected wavelength selected from the group consisting of near infrared light wavelengths and visible red light wavelengths, at a predetermined energy intensity, for a preset time duration, and at a predetermined pulse frequency, collectively on-demand sufficient to penetrate through the subject's skull and to pass into the brain;

wherein:

(A) said first configured irradiation unit is positioned to direct light energy to a first region of the brain comprising the OZ position of the primary visual cortex;

(B) said second configured irradiation unit is positioned to direct light energy to a second region of the brain comprising the CZ position of the primary sensorimotor cortex;

(C) said third configured irradiation unit is positioned to direct light energy to a third region of the brain comprising the C3 position of the primary motor cortex; and

(D) said fourth configured irradiation unit is positioned to direct light energy to a fourth region of the brain comprising the C4 position of the primary motor cortex.

### DESCRIPTION OF THE DRAWINGS

The present invention may be better understood and more readily appreciated when taken in conjunction with the accompanying drawings, in which:

FIG. 1 illustrates a perspective view of a preferred embodiment of the system of the present invention;

FIG. 2 illustrates another perspective view of a preferred embodiment of the system of the present invention;

FIG. 3 illustrates a front view of a preferred embodiment of the system of the present invention;

FIG. 4 illustrates a back view of a preferred embodiment of the system of the present invention;

FIG. 5 illustrates a side view of a preferred embodiment of the system of the present invention;

FIG. 6 illustrates a top view of a preferred embodiment of the system of the present invention;

FIG. 7 illustrates a bottom view of a preferred embodiment of the system of the present invention;

FIG. 8 illustrates a perspective view of a preferred embodiment of the system of the present invention applied to a subject;

FIG. 9 illustrates another perspective view of a preferred embodiment of the system of the present invention applied to a subject;

FIG. 10 illustrates another perspective view of a preferred embodiment of the system of the present invention applied to a subject;

FIG. 11 illustrates another perspective view of a preferred embodiment of the system of the present invention applied to a subject;

FIG. 12 illustrates a perspective view of an alternative embodiment of the system of the present invention having an intranasal unit applied to a subject;

FIG. 13 illustrates another perspective view of an alternative embodiment of the system of the present invention having an intranasal unit applied to a subject;

FIG. 14 illustrates another perspective view of an alternative embodiment of the system of the present invention having an intranasal unit applied to a subject;

FIG. 15 illustrates another perspective view of an alternative embodiment of the system of the present invention having an intranasal unit applied to a subject;

FIG. 16 illustrates a perspective view of a preferred intranasal unit used in an alternative embodiment of the system of the present invention;

FIG. 17 illustrates another perspective view of a preferred intranasal unit used in an alternative embodiment of the system of the present invention; and

FIG. 18 illustrates another perspective view of a preferred intranasal unit used in an alternative embodiment of the system of the present invention.

### DETAILED DESCRIPTION OF THE INVENTION AND PREFERRED EMBODIMENTS

The present invention relates to a wearable device to improve reflex-based abilities in sports and athletics. Preferably, the device improves one or more of a person's spatial and visual perception, target identification and hand-eye coordination, leading to quicker and more accurate movement responses. The device uses photobiomodulation (PBM), preferably delivering near infrared light at about 810 nm. Other adjustable parameters for the device may preferably include the location of the light generating unit, pulse frequencies, and phase synchrony.

Components of the Preferred System/Apparatus

The system and apparatus of the present invention preferably comprises at least the following component parts:

(1) a portable hollow casing;

(2) one or more light generating units which are housed and contained within the interior spatial volume of the hollow casing;

(3) a source of electrical current;

(4) a process controller assembly; and

(5) optionally, a smart phone, tablet computer or other computing device.

These components may preferably be electrically linked together by at least one connector for transfer of direct electrical current from the source of electrical current to the controller assembly, and at least one connector for conveyance of direct electrical current from the controller assembly to the light generating unit.

1. Portable Hollow Casing

The present invention includes at least one portable hollow casing having fixed dimensions, a sized internal spatial volume and an external surface configuration suitable for application to the subject. The intended purposes and goals of the portable casing are twofold: (i) to serve as a containment chamber that is configured for easy application to the subject; and (ii) to act as a molded lens that reflects and directs emitted light waves to the subject.

Preferably, the portable casing may be constructed and formed of a light transmitting material over at least a portion of its external surface, and will encompass that volumetric zone intended for housing and containment of at least one light generating unit. By definition, such light transmitting material includes and encompasses transparent, translucent and opaque matter. However, in most instances, a completely clear and transparent matter is preferred.

2. Light Generating Unit(s)

The light generating unit will be able to deliver therapeutic light at wavelengths that include but are not necessarily limited to the following: (i) in the visible color spectral ranges, the visible red light wavelengths ranging between about 620-780 nm; and (ii) in the non-visible spectral ranges, the near-infrared light wavelengths ranging between about 780-1400 nm. In addition, the generated light energy waves and particles may alternatively be: (i) either coherent (as in lasers) or non-coherent (as in non-laser light emitting diodes (LEDs); (ii) be either pulsing or non-pulsing (continuous wave) in delivery; (iii) be either constant or non-constant in intensity; (iv) be either uniform or non-uniform in phase; (v) polarized and non-polarized; and (vi) have a regular or irregular flux.

Any conventionally known means for generating electromagnetic radiation or articles for propagating radiant energy are acceptable for use in the present apparatus. In the majority of embodiments, it is intended and expected that either a low level laser unit or a LED will be employed as the light generating unit(s) for irradiating purposes.

3. Source of Electric Current

It is preferred that a portable and replenishable source of on-demand direct electrical current exist as a component part of the apparatus and system of the present invention. The therapeutic treatment system and method provided by the instant invention is intended to deliver a specific energy dosage (measured in Joules), which is a function of power (in wattage) and time (in seconds), and which is deemed to be efficacious for each therapeutic treatment.

The power supply typically will convey energy in the form of direct electric current. Adequate quantities of electric current can be repeatedly conveyed from, for example, a single battery source or from a combination of several dry cells joined together in series or parallel. In some other desirable embodiments, the source of electric power will be in the form of a rechargeable power bank, a direct current battery unit (rechargeable from ordinary household alternating current receptacles) or as alternating current (AC) via a power adaptor. It is expected and intended that there will be several alternative embodiments with different combinations of these components and which would be suitable for different configurations of power, energy dosage and treatment time.

As to positioning, in some preferred embodiments, the power source is a discrete entity which is held and contained entirely within the internal confines of the controller assembly. In other preferred embodiments, however, the source of electric current can be a self-contained, separate and free standing unit which is in electrical communication with the controller assembly via an electrical cable and connector module linkage, such as a portable and rechargeable power bank. In an alternative embodiment, the source of electrical current is obtained by plugging the system and apparatus into the local electrical grid via a power adaptor.

4. Process Controller Assembly

The process controller assembly is a portable unit component having at least three structural features:

(i) A receiving circuit for receipt of such electrical current as is transferred to the controller assembly from the electrical current source;

(ii) A central processing unit (CPU) for controlling and directing the flow of such electrical current as is received by the controller assembly over time; and

(iii) A delivery circuit for delivering direct electrical current from the controller assembly to the light generating unit(s).

It is intended and expected that the process controller assembly will be electrically linked to other essential components of the apparatus and thus typically will also have:

(a) at least one connector for transfer of direct electrical current from the source of electrical current to the controller assembly; and

(b) at least one connector for conveyance of direct electrical current from the controller assembly to the light generating unit(s).

These connectors typically are formed as insulated copper wire cables and jack modules that allow for quick and easy linkage and electrical communication with both the electrical current source and the light generating unit(s).

It is intended and expected that any conventionally known and interchangeable electric cables and connectors will be used to link the controller assembly to the irradiation lens. This also provides a distinct advantage and benefit to the user, namely the option to exchange one configured irradiation lens (able to transmit light at a first wavelength) for another irradiation lens (able to transmit light at a second and different wavelength), and thereby permits the use of different lasers and alternative light emitting diodes able to deliver different wavelengths of visible and invisible light energy with one single controller assembly.

In some preferred embodiments, the source of electrical current lies internally and is contained within the interior spatial volume of the controller assembly, and appears as an electric battery (dry cell or rechargeable unit). In this instance, the controller assembly also has a socket adapted for the attachment of an insulated copper wire cable and modular jack connector, whose other end is joined to the light generating unit disposed within the hollow casing.

The central processing unit (“CPU”) of the controller assembly is preferably able to regulate light energy with respect to many different parameters including but not limited to: wavelength, coherency/synchrony, energy (Joules (J)), Power (Watts (W) or milliwatts (mW)) or irradiance (W/cm²), radiant exposure (J/cm²), exposure time (seconds), pulse mode (continuous or pulse), frequency (Hertz (Hz)), duty cycle (percentage), fraction protocol (number of patient treatment sessions), light beam size (area of landed beam), and light beam penetration (delivery) distance.

The process controller assembly will not operate in the absence of a source of electrical current. In addition, the controller assembly, besides preferably switching off the unit after a predetermined time, is a circuitry which provides power to drive the light generating unit(s) properly and efficiently. The controller also ensures that the power delivered to the light generating unit(s) is consistent. It therefore desirably monitors the battery strength where the source is a power bank or battery, and switches off the unit if the power bank or battery is unable to supply sufficient power to drive the circuitry properly.

In a preferred embodiment, the controller is part of the headset assembly. Alternatively, the controller is detached from the headset but connected via a cable for communication.

5. Smart Phone, Tablet Computer or Other Computing Device

In one alternative embodiment, the function of the controller assembly is controlled, in whole or in part, by smartphone, smartwatch, tablet computer, laptop computer, desktop computer or any appropriate computing device. The smart phone, for example, may operate on one of the more popular mobile platforms. The light generating unit(s) could be connected via a cable or wirelessly to the smart phone. The smart phone carries a downloadable software application that would largely duplicate the software functions in the controller assembly. A modified attachment containing interface processing software in a computer chip will provide a physical connection between the existing controller and the proprietary smart phone platform. The software application will also contain more software controls and graphic interfaces. Alternatives to the smart phone include a smartwatch, tablet computer, laptop computer, desktop computer or any appropriate computing device with the software application downloaded thereon.

In yet another alternative embodiment, the controller assembly works in combination with smartphone, smartwatch, tablet computer, laptop computer, desktop computer or any appropriate computing device. In particular, the computing device has downloaded thereon a software application which can: (i) turn the controller assembly on and off; and/or (ii) transmit instructions to the controller assembly to adjust the light energy parameters of each individual light generating unit, including but not limited to wavelength, coherency/synchrony, energy (Joules (J)), Power (Watts (W) or milliwatts (mW)) or irradiance (W/cm²), radiant exposure or dose or fluence density (J/cm²), exposure time (seconds), pulse mode (continuous or pulse), frequency (Hertz (Hz)), duty cycle (percentage), fraction protocol (number of patient treatment sessions), light beam size (area of landed beam), and light beam penetration (delivery) distance.

Furthermore, the computing device can serve as a system interface where a user enters instructions through the interface to turn the controller assembly on and off and/or adjust the light energy parameters of each individual light generating unit. Instructions may be entered by any known input component such as a touch screen, mouse, keypad, keyboard, microphone, camera or video camera. Once the user inputs instructions into the system interface, instructions are transmitted to the controller assembly which then adjusts the parameters of the light energy being delivered to by the light generating units.

In these embodiments, any conventionally known and interchangeable electric cables and connectors can be used to link the computing device to the controller assembly. Alternatively, the computing device may communicate with the controller assembly by wireless means. Connections between any of these components are implemented using appropriate wired or wireless communications via protocols such as BLUETOOTH™, Wi-Fi, Near Field Communications (NFC), Radio Frequency Identification (RFID), 3G, Long Term Evolution (LTE), Universal Serial Bus (USB) and other protocols and technologies known to those skilled in the art.

Preferred Targets for the System of the Present Invention

In accordance with the inventor's research, the preferred targets for system of the present invention include the primary visual cortex, cerebellum, specific regions of the primary sensorimotor cortex, hippocampus and entorhinal cortex (EC)

Hippocampus and Entorhinal Cortex (EC)

The inventor has deemed the hippocampus and entorhinal cortex (EC) as being included as preferred targets for PBM in the present invention. Spatial perception and positioning sense may be determined at least in part by place cells in the hippocampus and grid cells in the entorhinal cortex of the brain. The hippocampus and EC may combine to code spatial cognition and map the position of the body at each moment in time in relation to the environment.

To improve spatial perception, one needs to recognize that the brain is plastic. Plasticity of the brain includes changes in neuronal connectivity. The inventor's research shows that pulsed PBM can change the connectivity of the brain. The effect on connectivity has led the inventor to propose that the direct projection from the olfactory bulb (positioned just above the nasal cavity) to the EC would be a preferred connection between intranasal PBM and hippocampus.

Furthermore, the conditioning for “muscle memory” involves the activities of the hippocampus and other relevant parts of the brain to encode new movement memories. The plastic nature of the brain allows it to create and consolidate the neuronal connections to enable this. As mentioned above, the inventor's research shows that pulsed PBM can improve the connectivity of the brain.

Based on the foregoing, the inventor proposes that using intranasal PBM would be a preferred way to irradiate the EC and the hippocampus. Therefore, the system of the present invention preferably has an intranasal PBM applicator, which can be a removable option.

Primary Visual Cortex and Cerebellum

The inventor has also deemed the primary visual cortex as a preferred target, at least in part for improved visual processing. The primary visual cortex, often called V1, is the structure that consciously processes visual stimuli. The area is normally labelled as V1 or described as OZ under the 10-20 convention.

Furthermore, the inventor has decided that the cerebellum is also a preferred target. The cerebellum provides a “forward model” used for control of movement, a role in hand-eye coordination. In this role, the forward model would generate time-specific signals predicting the motion of each motor effector, essential for predictive control of, for example, eye and hand movements.

The quality of reflex action also seems to be affected by the health of the cerebellum, which located just below and adjacent to the primary visual cortex. Uncontrolled movements in essential tremors can be associated with a dysfunctional cerebellum.

The H-reflex is the electrical analog of the spinal stretch reflex (SSR). H-reflex conditioning seems to depend on a hierarchy of brain and spinal cord plasticity to which the cerebellum may contribute. The inventor believes that the cerebellum is important in the maintenance of reflex conditioning that has already occurred.

The cerebellum is also reported to grow in volume in response to motor skill learning, and it may be related to the acquisition of motor coordination.

Based on the inventor's research, a PBM device to stimulate the subject's visual processing, hand-eye coordination, reflex conditioning, and motor skill learning and performance would preferably be targeted to the primary visual cortex, which is the OZ position. Due to light scattering upon tissue penetration, light from a light generating unit on OZ would cover both the primary visual cortex and the cerebellum.

Primary Sensorimotor Cortex

The inventor's research has also led to the Primary Sensorimotor Cortex (referred as “M1” here) being another preferred target for the system of the present invention. Each part of our body is represented in a distinct area of the M1. Learning a new motor skill would develop a motor program in the M1, supported by synergies with other connected areas of the brain responsible for executing that motor skill. Motor programs and synergies develop in response to learning and plasticity.

Movements from the M1 area of the brain are executed through neural impulses that pass down to the spinal cord and control the execution of movement. The areas of the sensorimotor cortex that lead to movements in specific parts of the body is represented by different locations on the sensorimotor cortex. For hand movements, the locations are approximately in the C3 (left) and C4 (right) areas identified under the 10-20 convention.

Optimal function of the sensorimotor cortex is highly desirable in sports. It determines how well and accurately the athlete is able to quickly and accurately attack targets. Training the sensorimotor rhythm (SMR) at 12 to 15 Hz oscillatory frequencies could improve focus and attention as well as enhance sensorimotor functions.

The inventor believes that PBM can optimize M1 neurons for plasticity by increasing blood flow to the stimulated area, increasing expression of growth factors, and upregulating mitochondrial production. Collectively, these mechanisms create new neuronal connections. The system of the present invention may preferably be used right before a training session to prime the M1 for learning and training. It can also be used during competition to increase performance.

Covering the primary sensorimotor cortex (M1), the system of the present invention preferably has three light emitting diodes (LED) placed along the M1 in locations corresponding to positions CZ, C3, and C4 of an electroencephalography (EEG) montage. CZ is at the top of the head, and C3 and C4 are about 20% of the distance between the preauricular point and CZ.

Simultaneous Activation of Preferred Targets by PBM

In the present invention using PBM, it is beneficial and preferred to have simultaneous activation of the relevant sections of the primary sensorimotor cortex, the primary visual cortex and the hippocampus. Obtaining the right plasticity of the brain consolidated with repetitive training can result in improved athletic performance.

In accordance with the inventor's research, it is expected that when photons of the relevant wavelength are delivered simultaneously to the sensorimotor cortex, visual cortex, cerebellum and hippocampus during training, we can expect to develop improved reaction time and hand-eye coordination in sports performance. In PBM, apart from the wavelengths, the power and exposure time leading to the energy dosage are also important in the determining the outcomes.

Preferred Frequency of PBM

The pulse rates influence different brain wave frequencies that are associated with different brain states. For example, gamma brain waves (30 Hz and above) have been associated with transient higher order brain processing, memory functions and even reduction of Alzheimer's Disease markers. Slow waves (slower than 8 Hz) have been associated with disengagement with the surroundings and sleep. Acute learning and plasticity may be activated at high gamma frequencies and quality performance may be expressed at low gamma. To train SMR rhythm, one could aim for 12 to 15 Hz in the sensorimotor cortex.

The inventor has considered the option of improving memory formation by encoding the PBM with fast gamma (60-80 Hz) or memory retrieval with slow gamma (40-50 Hz), both of which are within the scope of the present invention. The 80 Hz brain frequency can be harmonically coupled with 40 Hz frequency. Alternatively, the LEDs can be pulsed at 12 to 15 Hz for SMR training to improve focus and attention, essential for sports performance.

Preferred Embodiments of the System of the Present Invention

In one preferred embodiment, the system of the present invention comprises the following features:
1. 3 LED diodes with a wavelength of 810 nm (power: 100 mW/cm²) placed over M1 regions corresponding to CZ, C3, and C4 with a pulse frequency of 14 Hz. As an option to hold the placements, the diodes over the primary motor areas could be part of an attachable band;
2. 1 LED diode placed over the primary visual cortex (which also irradiates the cerebellum) with 810 nm (power: 30 mW/cm²) with an option to select pulse frequencies of 14 Hz;
3. 1 detachable intranasal LED 810 nm (power: 25 mW/cm²) with an option to select pulse frequency of 14 Hz
4. Power source would be a power bank; and
5. The intranasal unit could pulse out-of-phase (asynchronous) with the other modules.

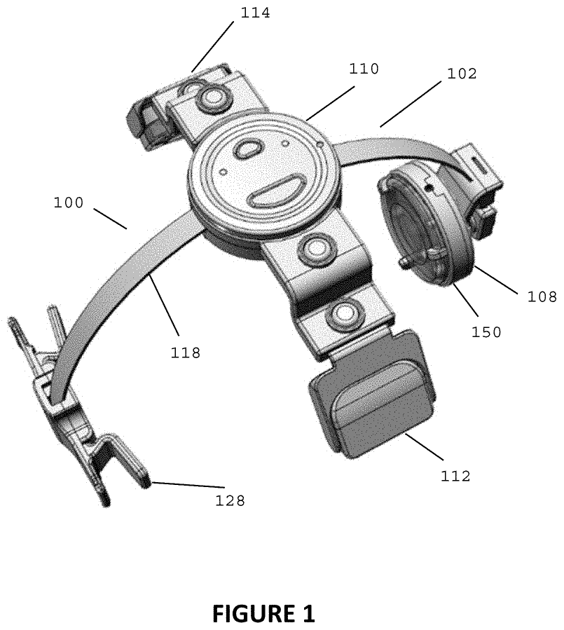

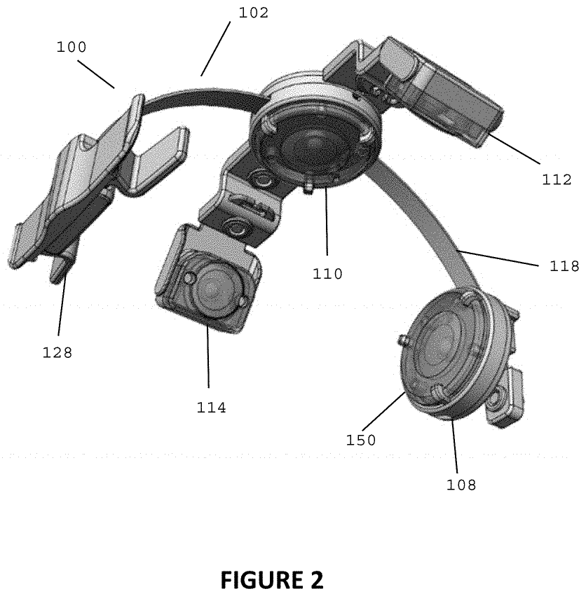

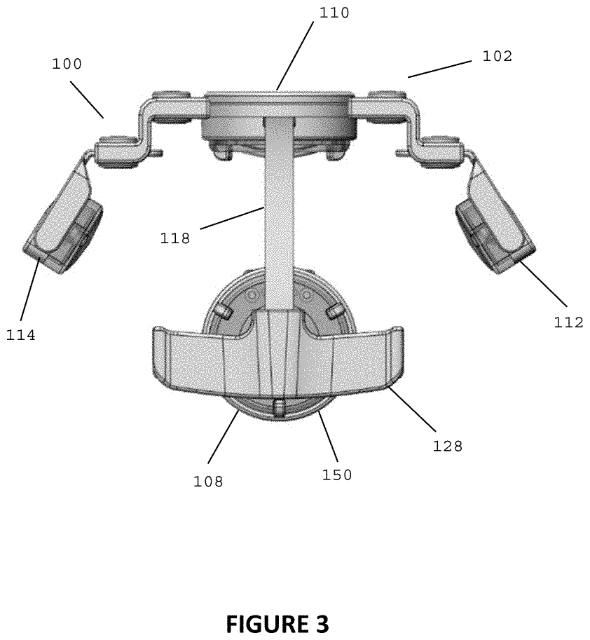

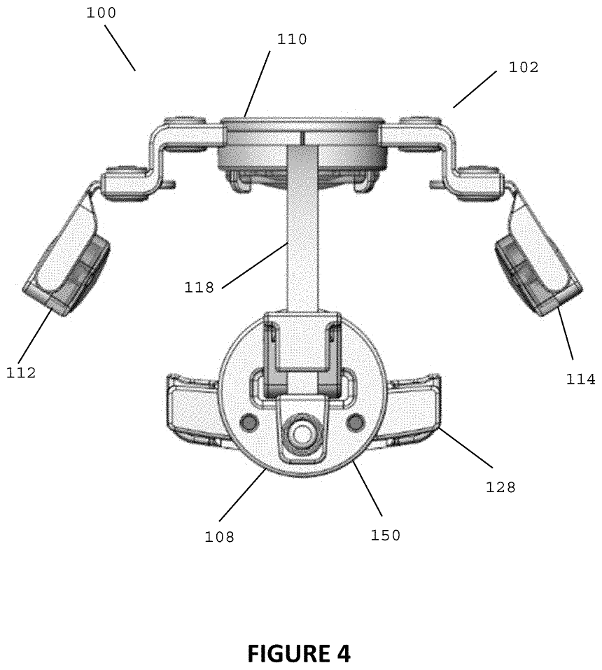

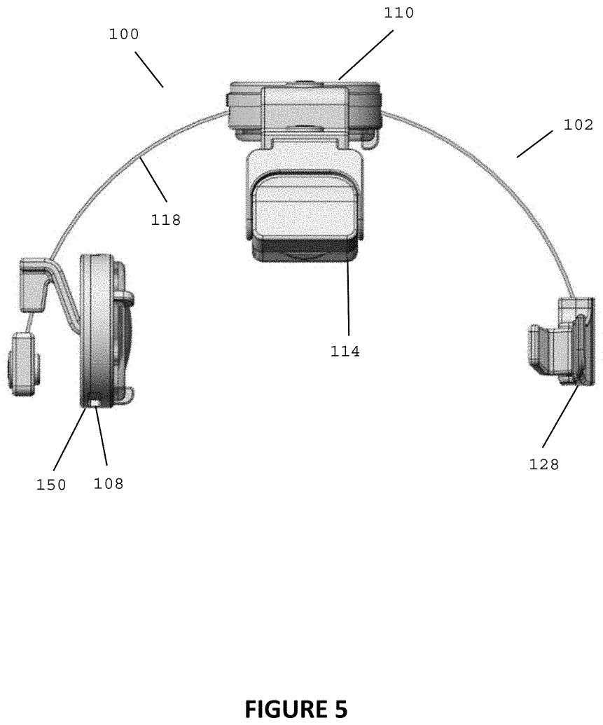

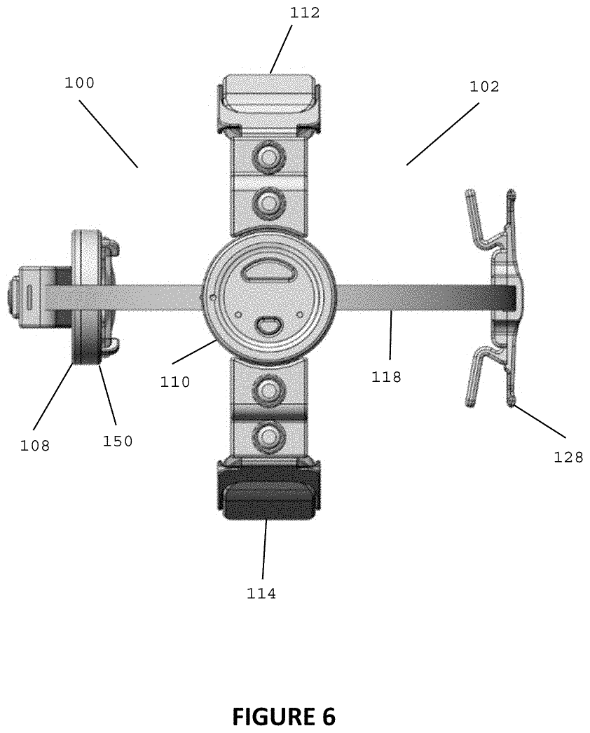

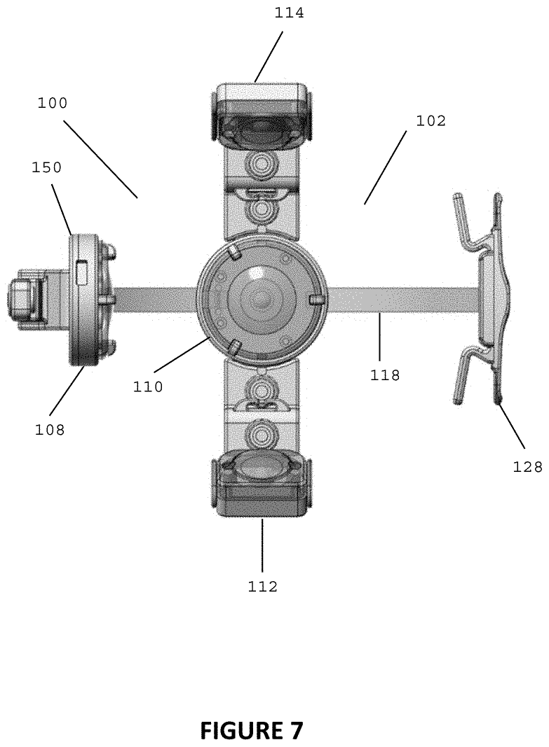

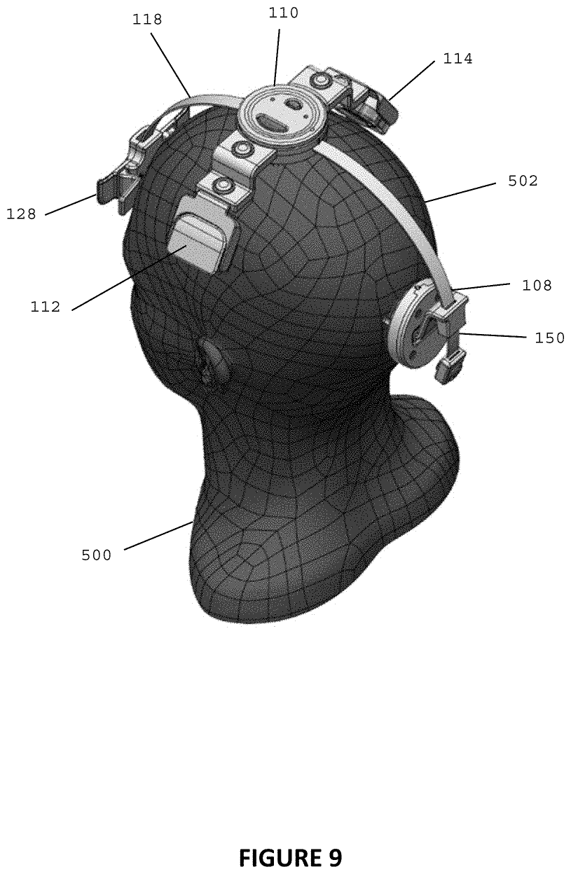

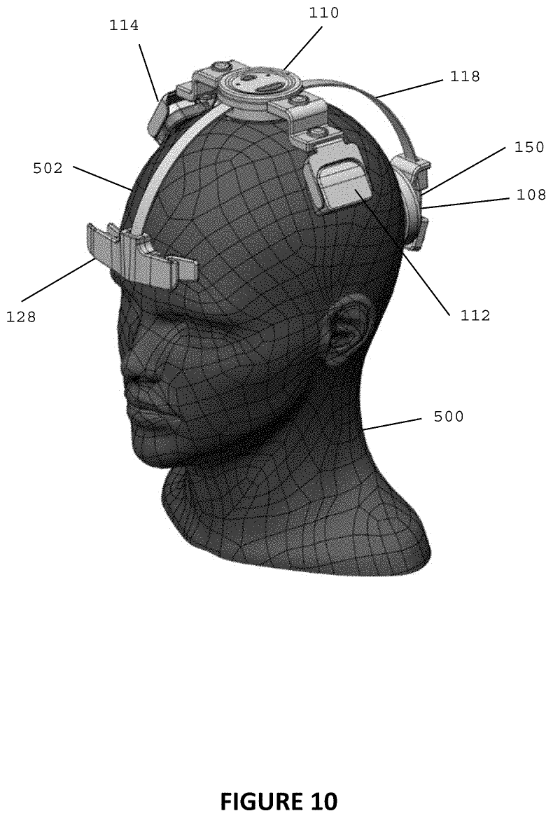

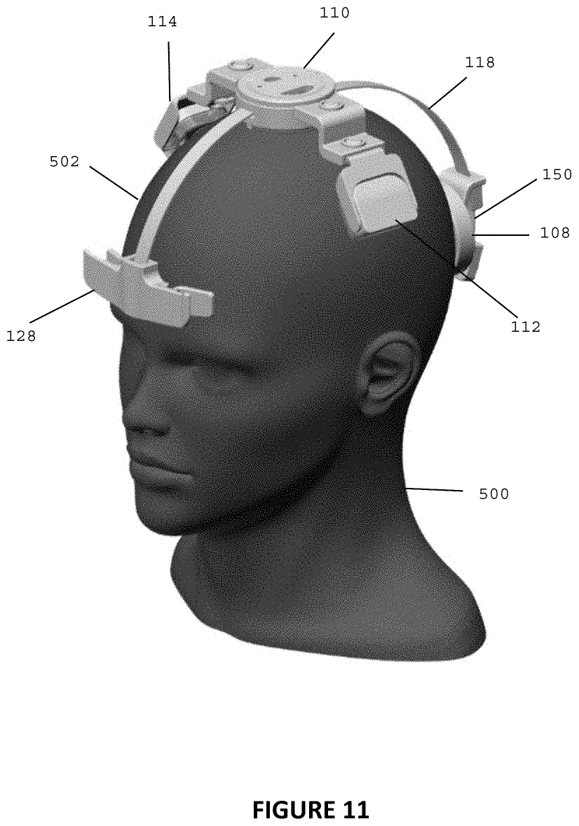

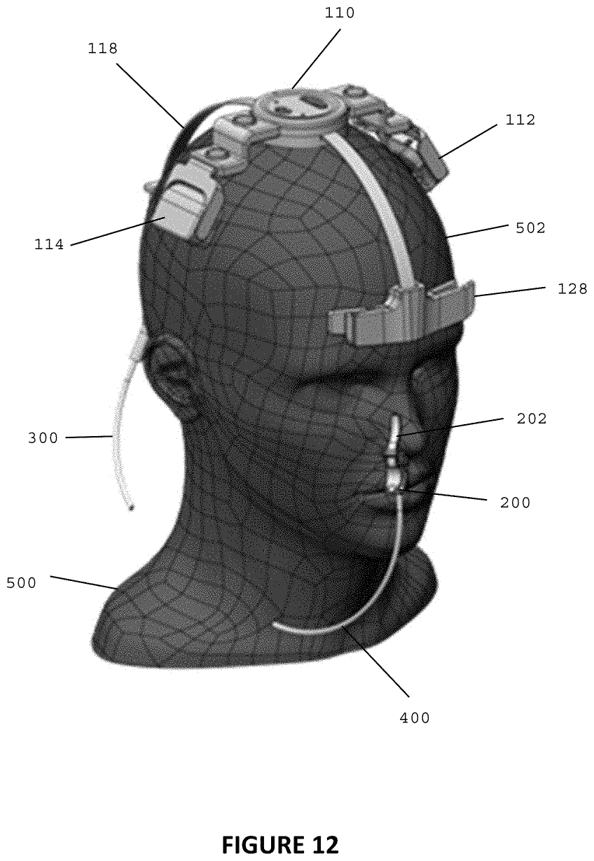

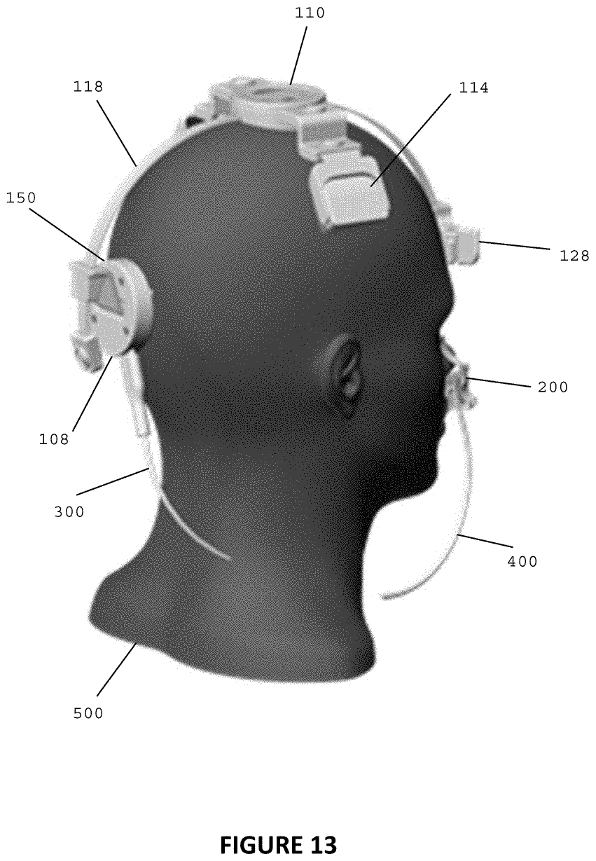

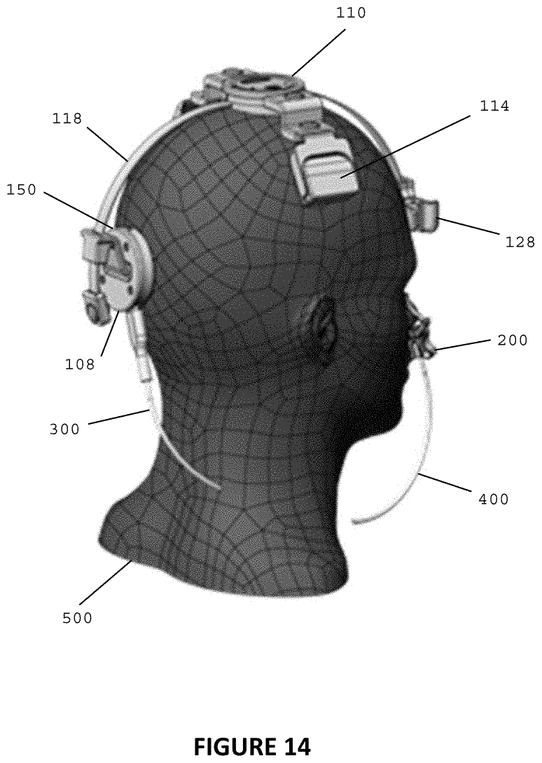

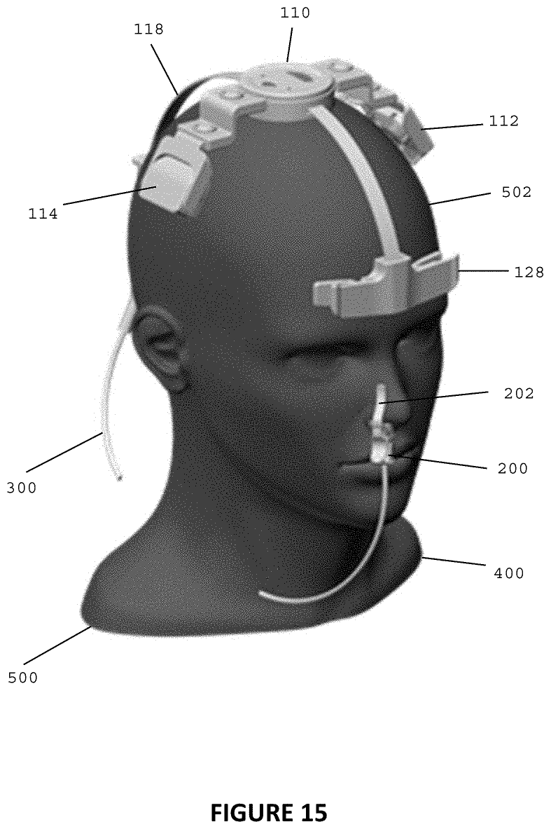

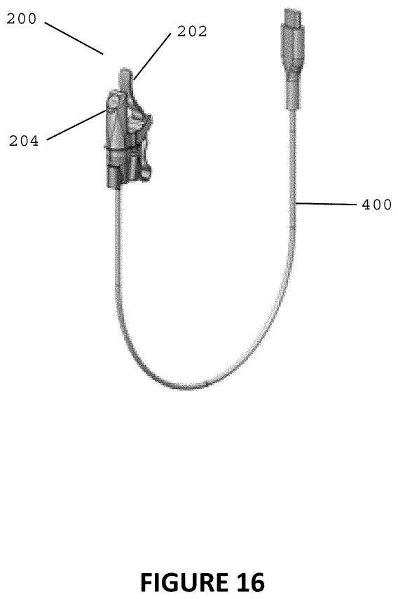

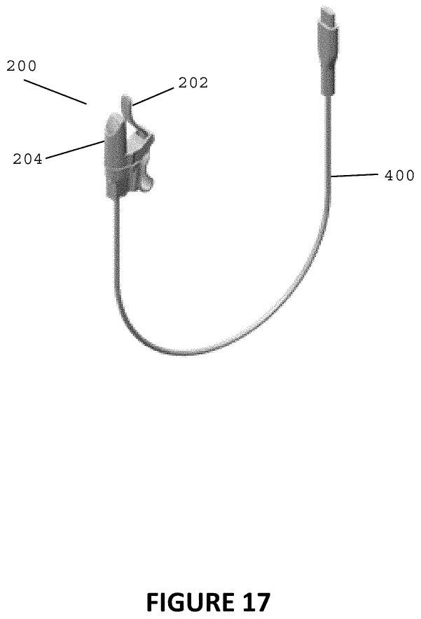

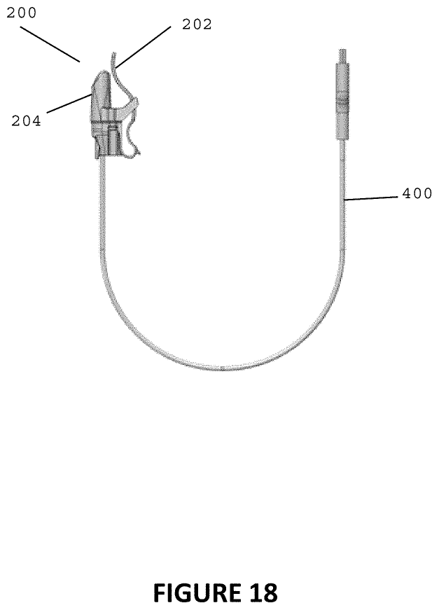

As shown in FIGS. 1 to 11, the present invention provides a preferred embodiment of an apparatus 100 having a transcranial light therapy headset 102. Optionally, as can be seen in FIGS. 12 to 18, there is an intranasal unit 200 as well.

A controller assembly 150 can serve as a power source and central processing unit for both the transcranial headset 102 and intranasal unit 200. In the preferred embodiment shown in FIGS. 1 to 18, the controller assembly 150 is located on the transcranial headset 102. In alternative embodiments, the controller assembly 150 is a separate unit which can communicate with the transcranial headset 102 and intranasal unit 200.

Referring to FIGS. 1 to 11, the headset 102 comprises one or more configured irradiation units 108, 110, 112 and 114, each of the configured irradiation units 108, 110, 112 and 114 including a portable hollow casing having fixed dimensions, a sized internal spatial volume, and an external surface configuration suitable for application to the skull 502 of the subject 500.

The portable casing comprises: (i) a light energy transmitting material which forms at least a portion of the configured external surface for said hollow casing, and (ii) at least one light generating unit entirely housed and contained within said internal spatial volume of said hollow casing and which is capable of generating light energy of at least one preselected wavelength selected from the group consisting of near infrared red light wavelengths and visible red light wavelengths, at a predetermined energy intensity and for a preset time duration on-demand sufficient to penetrate through the skull 502 and to pass into the brain.

A frame 118 is provided in the headset 102 to support the configured irradiation units 108, 110, 112 and 114 and to adapt the headset 102 for at will placement of the light transmitting external surface of the configured irradiation units 108, 110, 112 and 114 at a fixed position and desired irradiation direction on the skull 502. Support structure 128 is preferably provided to help secure the headset 102 to the skull 502 and to make the headset 102 more comfortable for the subject 500 to wear.

In the preferred embodiment shown in FIGS. 1 to 7, the frame 118 supports four configured irradiation units 108, 110, 112 and 114, and each configured irradiation unit 108, 110, 112 and 114 has light generating unit(s).

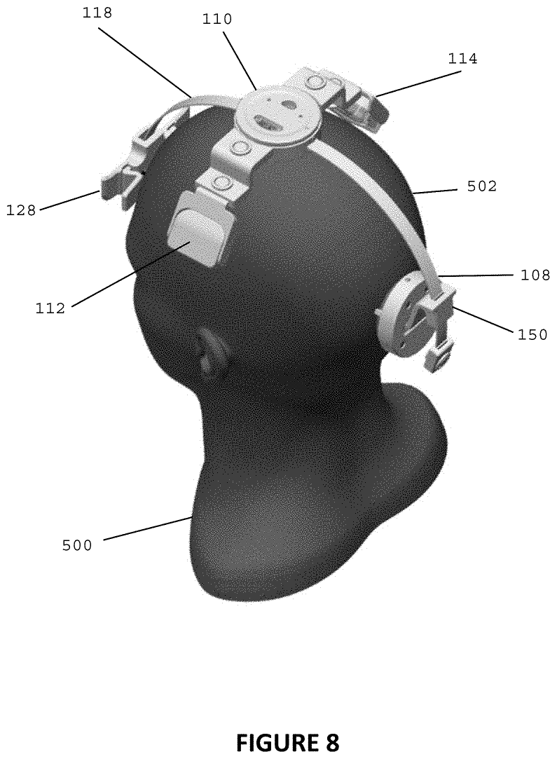

The four configured irradiation units 108, 110, 112 and 114 are positioned in the headset 102 such that they target specific locations. In the preferred embodiment shown in FIGS. 8 to 11, the four configured irradiation units 108, 110, 112 and 114 are positioned to target the following parts of the subject 500:

(A) a first region of the brain comprising the OZ position of the primary visual cortex;

(B) a second region of the brain comprising the CZ position of the primary sensorimotor cortex;

(C) a third region of the brain comprising the C3 position of the primary sensorimotor cortex; and

(D) a fourth region of the brain comprising the C4 position of the primary sensorimotor cortex.

As can be seen in FIGS. 12 to 18, the preferred system of the present invention optionally comprises an intranasal light therapy unit 200 which includes a nose clip 202. The nose clip 202 holds a configured irradiation lens 204 inside one of the nostrils of the subject. The configured irradiation lens 204 includes a portable hollow casing having fixed dimensions, a sized internal spatial volume, and an external surface configuration suitable for application to the interior of the nostrils. The portable casing comprises: (i) a light energy transmitting material which forms at least a portion of the configured external surface for said hollow casing, and (ii) at least one light generating unit entirely housed and contained within said internal spatial volume of said hollow casing and which is capable of generating light energy of at least one preselected wavelength selected from the group consisting of near infrared red light wavelengths and visible red light wavelengths, at a predetermined energy intensity and for a preset time duration on-demand sufficient to penetrate through the nasal tissues and to pass into the brain.

A first connector 300 may be in electrical communication with the configured irradiation units 108, 110, 112 and 114 of the transcranial headset 102. A second connector 400 may be in electrical communication with the configured irradiation lens 204 of the intranasal light therapy unit 200. This allows for on-demand conveyance of direct electrical current from a power source, such as through the controller assembly 150, to the light generating units in the configured irradiation units 108, 110, 112 and 114, as well as the light generating unit(s) of configured irradiation lens 204 in the intranasal light therapy unit 200.

Experimental Section

Objective

The objective of this test was to observe the effects of a preferred embodiment of the system of the invention with preferred specifications (training SMR rhythm at 14 Hz) on an activity, specifically golf putting.

Subjects

Four healthy male participants, aged 46, 50, 55 and 58 years old, volunteered for this test. All were not regular golfers.

Procedure

The golf putting facility chosen for the test was an indoor green of a larger indoor golf practice facility that is isolated from outside environmental noise. The participants were required to stand 2.5 meters from the edge of a hole of 10.8 cm diameter.

The procedure was conducted in the following stages:

1. For orientation (data not used for analysis), the participants putted in series of 10 putts each, until they established a cumulative 30% average for successful putts.

2. During the SMR training, the two participants of ages 50 and 58 carried out a series of 40 putts wearing the system of the invention, every alternate day. The two participants of ages 46 and 55 were the control group who carried out the same activity but without the use of the invention. This was carried out over a 2-week period. The distance between the edge of the hole and the edge of the ball after a putt was measured each time. At the end of the day, the mean distances were measured for each participant.

3. At the end of the second week, the results were collected and analyzed.

Results

The mean distance of the active SMR group in the pretest and posttest using the system of the invention was 32.15 cm and 20.62 cm, respectively. The improvements had a mean distance of 11.53 cm or 35.86%. The control group distance was 26.67 cm and 24.28 cm, respectively. The improvements in the control group had a mean distance of 2.39 cm or 8.96%.

The active SMR group using the system of the present invention showed significantly superior improvement in golf putts over the control group. This data suggest that there is potential for use of this invention to improve hand-eye coordination in a target-based sports.

The scope of the claims should not be limited by the preferred embodiments set forth in the examples, but should be given the broadest interpretation consistent with the description as a whole.

1. A self-administrable system for improving athletic performance of a subject, said system comprising:
configured irradiation units comprising a first, a second, a third and a fourth configured irradiation unit, each of said first, second, third and fourth configured irradiation units comprising a portable hollow casing having fixed dimensions, a sized internal spatial volume and an external surface configuration suitable for application to the skull, said portable hollow casing of each configured irradiation unit being comprised of:
(i) a light energy transmitting material which forms at least a portion of the configured external surface for said hollow casing of each configured irradiation unit; and
(ii) at least one light generating unit housed and contained within said internal spatial volume of said hollow casing of each configured irradiation unit and which is capable of generating light energy of at least one preselected wavelength selected from the group consisting of near infrared light wavelengths and visible red light wavelengths, at a predetermined energy intensity, for a preset time duration, and at a predetermined pulse frequency, collectively on-demand sufficient to penetrate through the skull and to pass into the brain, whereby said first, second, third and fourth configured irradiation units can emit light energy after application to the skull and achieve passage of said emitted light energy through the skull into at least one portion of the brain in-vivo;
a frame adapted for support of said first, second, third and fourth configured irradiation units and for at will placement of said light transmitting external surface of said first, second, third and fourth configured irradiation units at a fixed position and desired irradiation direction on the skull;
a portable controller assembly able to control on-demand delivery of light energy from said configured irradiation units into at least one portion of the brain in-vivo, said controller assembly including:
(a) a portable and replenishable power source of on-demand direct electrical current,
(b) a central processing unit for controlling and directing the flow of such direct electrical current,
(c) at least one connector in electrical communication with the power source for on-demand conveyance of direct electrical current to the central processing unit, and
(d) at least one connector in electrical communication with the configured irradiation units for on-demand conveyance of direct electrical current from said central processing unit to said light generating units;
wherein:
(A) said first configured irradiation unit is positioned to direct light energy to a first region of the brain comprising the OZ position of the primary visual cortex;
(B) said second configured irradiation unit is positioned to direct light energy to a second region of the brain comprising the CZ position of the primary sensorimotor cortex;
(C) said third configured irradiation unit is positioned to direct light energy to a third region of the brain comprising the C3 position of the primary sensorimotor cortex; and
(D) said fourth configured irradiation unit is positioned to direct light energy to a fourth region of the brain comprising the C4 position of the primary sensorimotor cortex.

2. The system of claim 1, said system further comprising:
a configured irradiation lens including:
a portable hollow casing having fixed dimensions, a sized internal spatial volume, and an external surface configuration suitable for in-vivo insertion into the nasal cavity space of a nostril without causing substantial impairment to the subject's ability to breathe and without invading the nasal tissues of the living subject, said portable casing of said configured irradiation lens being comprised of:
(i) a light energy transmitting material which forms at least a portion of the configured external surface for said hollow casing of said configured irradiation lens,
(ii) at least one light generating unit housed and contained within said internal spatial volume of said hollow casing of said configured irradiation lens and which is capable of generating light energy of at least one preselected wavelength selected from the group consisting of near infrared light wavelengths and visible red light wavelengths, at a predetermined energy intensity and for a preset time duration on-demand sufficient to penetrate through the nasal tissues and to pass into the brain,
whereby said configured irradiation lens can emit light energy in any desired direction within the nasal cavity after in-vivo insertion and achieve passage of said emitted light energy from the nasal cavity into at least one portion of the brain in-vivo;
a self-administrable applicator means adapted for support of said configured irradiation lens and for at will placement of said light transmitting external surface of said configured irradiation lens at a fixed position and desired irradiation direction within a nostril adjacent to the internal lining of a subject's nasal cavity;
wherein said portable controller assembly is further able to control on-demand delivery of light energy from said configured irradiation lens.

3. The system of claim 2, wherein the configured irradiation lens is positioned to direct light energy to a fifth region of the brain selected from the group consisting of the olfactory bulb, entorhinal cortex and hippocampus.

4. The system of claim 2, wherein the configured irradiation lens pulses light energy out of phase with at least one of the first, second, third and fourth configured irradiation units.

5. The system of claim 1, wherein the light energy has a wavelength of about 810 nm.

6. The system of claim 1, wherein the light energy is pulsed at a frequency of about 30 to 50 Hz.

7. The system of claim 1, wherein the light energy is pulsed at a frequency of about 70 to 90 Hz.

8. The system of claim 1, wherein the light energy is pulsed at a frequency of about 12 to 15 Hz.

9. A self-administrable method for improving athletic performance of a subject, said method comprising the steps of:
obtaining a light energy-emitting apparatus comprised of:
configured irradiation units comprising a first, a second, a third and a fourth configured irradiation unit, each of said first, second, third and fourth configured irradiation units comprising a portable hollow casing having fixed dimensions, a sized internal spatial volume and an external surface configuration suitable for application to the skull, said portable hollow casing of each configured irradiation unit being comprised of:
(i) a light energy transmitting material which forms at least a portion of the configured external surface for said hollow casing of each configured irradiation unit; and
(ii) at least one light generating unit housed and contained within said internal spatial volume of said hollow casing of each configured irradiation unit and which is capable of generating light energy of at least one preselected wavelength selected from the group consisting of near infrared light wavelengths and visible red light wavelengths, at a predetermined energy intensity, for a preset time duration, and at a predetermined pulse frequency, collectively on-demand sufficient to penetrate through the skull and to pass into the brain,
whereby said first, second, third and fourth configured irradiation units can emit light energy after application to the skull and achieve passage of said emitted light energy through the skull into at least one portion of the brain in-vivo;
a frame adapted for support of said first and second configured irradiation units and for at will placement of said light transmitting external surface of said first and second configured irradiation units at a fixed position and desired irradiation direction on the skull;
a portable controller assembly able to control on-demand delivery of light energy from said configured irradiation lenses into at least one portion of the brain in-vivo, said controller assembly including:
(a) a portable and replenishable power source of on-demand direct electrical current,
(b) a central processing unit for controlling and directing the flow of such direct electrical current,
(c) at least one connector in electrical communication with the power source for on-demand conveyance of direct electrical current to the central processing unit, and
(d) at least one connector in electrical communication with the configured irradiation units for on-demand conveyance of direct electrical current from said central processing unit to said light generating units;
placing a transparent external surface of said first, second, third and fourth configured irradiation units at a desired fixed position adjacent to the skull of a subject such that light energy emitted by said first, second, third and fourth configured irradiation units will penetrate through the subject's skull and pass into at least one portion of the brain in-vivo; and
causing said light generating units of said positioned configured irradiation units to generate light energy of at least one preselected wavelength selected from the group consisting of near infrared light wavelengths and visible red light wavelengths, at a predetermined energy intensity, for a preset time duration, and at a predetermined pulse frequency, collectively on-demand sufficient to penetrate through the subject's skull and to pass into the brain;
wherein:
(A) said first configured irradiation unit is positioned to direct light energy to a first region of the brain comprising the OZ position of the primary visual cortex;
(B) said second configured irradiation unit is positioned to direct light energy to a second region of the brain comprising the CZ position of the primary sensorimotor cortex;
(C) said third configured irradiation unit is positioned to direct light energy to a third region of the brain comprising the C3 position of the primary sensorimotor cortex; and
(D) said fourth configured irradiation unit is positioned to direct light energy to a fourth region of the brain comprising the C4 position of the primary sensorimotor cortex.

10. The method of claim 9, wherein said light energy-emitting apparatus further comprises:
a configured irradiation lens including:
a portable hollow casing having fixed dimensions, a sized internal spatial volume, and an external surface configuration suitable for in-vivo insertion into the nasal cavity space of a nostril without causing substantial impairment to the subject's ability to breathe and without invading the nasal tissues of the living subject, said portable casing of said configured irradiation lens being comprised of:
(i) a light energy transmitting material which forms at least a portion of the configured external surface for said hollow casing of said configured irradiation lens,
(ii) at least one light generating unit housed and contained within said internal spatial volume of said hollow casing of said configured irradiation lens and which is capable of generating light energy of at least one preselected wavelength selected from the group consisting of near infrared light wavelengths and visible red light wavelengths, at a predetermined energy intensity and for a preset time duration on-demand sufficient to penetrate through the nasal tissues and to pass into the brain,
whereby said configured irradiation lens can emit light energy in any desired direction within the nasal cavity after in-vivo insertion and achieve passage of said emitted light energy from the nasal cavity into at least one portion of the brain in-vivo;
a self-administrable applicator means adapted for support of said configured irradiation lens and for at will placement of said light transmitting external surface of said configured irradiation lens at a fixed position and desired irradiation direction within a nostril adjacent to the internal lining of a subject's nasal cavity;
wherein said portable controller assembly is further able to control on-demand delivery of light energy from said configured irradiation lens;
wherein said method further comprises:
placing a transparent external surface of said configured irradiation lens within a nostril at a desired fixed position adjacent to the internal lining of a subject's nasal cavity such that light energy emitted by said configured irradiation lens will penetrate through the subject's nasal tissues and pass into at least one portion of the brain in-vivo; and
causing said light generating units of said positioned configured irradiation lens to generate light energy of at least one preselected wavelength selected from the group consisting of near infrared light wavelengths and visible red light wavelengths, at a predetermined energy intensity and for a preset time duration on-demand sufficient to penetrate through the subject's nasal tissues and to pass into the brain.

11. The method of claim 10, wherein the configured irradiation lens is positioned to direct light energy to a fifth region of the brain selected from the group consisting of the olfactory bulb, entorhinal cortex and hippocampus.

12. The method of claim 10, wherein the configured irradiation lens pulses light energy out of phase with at least one of the first, second, third and fourth configured irradiation units.

13. The method of claim 9, wherein the light energy has a wavelength of about 810 nm.

14. The method of claim 9, wherein the light energy is pulsed at a frequency of about 30 to 50 Hz.

15. The method of claim 9, wherein the light energy is pulsed at a frequency of about 70 to 90 Hz.

16. The method of claim 9, wherein the light energy is pulsed at a frequency of about 12 to 15 Hz.
